(19) United States
(12) Patent Application Publication
(10) Pub. No.: US 2004/0033933 A1
(43) Pub. Date: Feb. 19, 2004

(54) Process for preparing composition comprising medicinal herb extract for preventing and curing arthritis and composition thereof

(76) Inventors: Nam Ki Lee, Seattle, WA (US); Jong Ho Lee, Gyoungki-do (KR); Moo Shin Park, Seoul (KR); Chul Hoon Kang, Gyoungki-do (KR); Nak Won Sohn, Seoul (KR); Hong Joon Ahn, Seoul (KR)

Correspondence Address: MERCHANT & GOULD PC, P.O. BOX 2903, MINNEAPOLIS, MN, 55402-0903, US

(21) Appl. No.: 10/333,376

(22) PCT Filed: Jul. 10, 2001
(86) PCT No.: PCT/KR01/01181

(30) Foreign Application Priority Data
Jul. 19, 2000 (KR) 2000/41421
Jul. 10, 2001 (KR) 2001/41125

(51) Int. Cl. A61K 31/00

(52) U.S. Cl. 514/1

(57) ABSTRACT

The present invention provides compositions containing herbal extracts for preventing and treating arthritis and a manufacturing method of the same. Particularly, pharmaceutical compositions extracted from Achyranthis roots and Atractylodes japonica roots are very effective against arthritis, and further, fermentative by-products of the compositions have excellent effects in treating and preventing arthritis in contrast to the existing artificial chemical treatment materials.

### TECHNICAL FIELD

The present invention relates to herbal extract compositions for treating and preventing arthritis effectively and processes for preparation of the same.

More particularly, the present invention relates to herbal extract-based pharmaceutical compositions comprising Achyranthis roots (oosle), Atractylodes japonica roots (changchul), their mixture and these fermented product which can treat arthritis by inhibiting the production of tumor necrosis factor a (TNF-α) and can regenerate osteoblast and cartilaginous tissue with anti-inflammation and collagen synthesis by promoting the production of transforming growth factor β (TGF-β) and interleukin and processes for the preparation thereof

### BACKGROUND ART

Arthritis is an inflammatory disease of joints and can be induced in various forms such as rheumatoid arthritis and joint inflammation related diseases.

Especially, among arthritis related diseases chronic polyarthritis has a feature of inflammation characteristic changes in synovial membrane of joint capsule' inner layer and induces edema and pain in the joints of whole body. In extreme cases, it is aggravated to a chronic disease so as to make a physically handicapped person. Besides, arthritis affection such as rheumatoid arthritis and so on is progressive and generates joint lesions (athropathy) like deformation and acampsia. Furthermore, the arthritis often causes a serious physical handicap when it is not treated properly and deteriorates continuously.

The direct mechanism of arthritis induction has not been discovered yet but active researches have been going on to find it out. Also, the therapeutic method for preventing such arthritis affection as rheumatoid arthritis and the like has not been reported. In addition, there are a lot of problems for the part of the treatment once the arthritis disease invades.

Conventionally, in order to treat such an arthritis, chemotherapeutic cure has been adopted by using various types of drugs that contain anti-inflammatory agent of steroid system such as cortisone and other adrenocortical hormones, anti-inflammatory agent of non-steroid system such as asprin, pyrroxicam and indomethacin, auric agent such as orothiomalic acid, anti-rheumatoid agent such as chloroquinone and D-phenisylamine, gout inhibitor such as colchicin and immunosuppressant such as cyclophosphamide, azathioprine, methotrexate and levamisol.

However, the above chemotherapeutic drugs cannot treat the disease fundamentally and produces a lot of adverse reactions of medicine in case that steroid hormones known as an arthritis drug are administered for the treatment

In addition, anti-inflammatory agent and the like have to be administered simultaneously since arthritis disease, especially chronic rheumatoid arthritis, gives a patient serious pain. In order to alleviate this pain and remove joint abscess, an antiphlogistic agent including aspirin, btazolin and the like has long been utilized as a treatment medicine widely. But it is difficult to administer the required amount of drug such as aspirin and so on for the treatment of arthritis continuously since this causes fatal damage on the human stomach.

Unfortunately, chemotherapeutic drugs shown previously have some disadvantages. Concretely, there are problems like side effect inhibiting long time administration, deficiency of anti-inflammation, lack of efficacy toward already provoked arthritis and the like. At the present time, indomethacin and ibuprofen which can soothe pains effectively during the treatment process of arthritis and various kinds of non-steroid antiphlogistic agent are prepared only for the administration.

Therefore, it is necessary to solve these problems and to develop a new pharmaceutical agent for alleviating acute inflammation symptom and pain sufficiently. Especially, a drug without side effect is strongly needed since it is usually administered for long time in order to treat arthritis such as rheumatoid arthritis and so on and most drugs adopted presently have some side effects in spite of individual differences.

Among the pharmaceutical drugs described above, certain kinds are troublesome because they are prescribed in the forms of intravenous injection or peritoneal injection. Besides, if these injections are repeated allergy, shock and the like occur and also hygienic problems can follow. Hence, safe edible drugs for treatment are required.

### DISCLOSURE OF THE INVENTION

To overcome the foregoing and other disadvantages and to satisfy the above needs, we, the inventors of the present invention, have developed a new pharmaceutical compositions.

It is an object of the present invention to provide a process for preparing an herbal extract composition for preventing and treating arthritis.

It is another object of the present invention to provide herbal extract compositions prepared by using the above process.

In order to accomplish the above objects, the present invention provides a process for preparing a herbal extract composition, which comprises the steps of; (1) extracting Achyranthis roots (oosle) and Atractylodes japonica roots (changchul) and (2) fermenting the herbal extracts.

Preferably, the process for preparing of the present invention comprises a further step; (3) concentrating the above herbal extracts after the fermentation step (2).

At that time, the solvent utilized for the extraction step (1) can be selected from the group consisting of distilled water, ethanol, methanol, propanol or butanol and this solvent mixture preferably. More preferably, the solvent is distilled water, ethanol or methanol.

In addition, Achyranthis roots and Atractylodes japonica roots are in dried herb state or in raw herb state preferably. The ratio of Achyranthis roots and Atractylodes japonica roots is preferably 0:5˜5:0 and more preferably 1:10˜10:1 and most preferably 1:2. In case that Achyranthis root is utilized in raw state, the ratio is estimated by a weight ratio and considering its water content.

Although both herb extracts of Achyranthis roots and Atractylodes japonica roots are effective for treating arthritis in the present invention, Achyranthis root content and its fermented product play the most important roles to have curing effects. Concretely, they prevent the activation of osteoclast by inhibiting the production of tumor necrosis factor α (TNF-α) and by interfering the activation of collagenase and the like and they also enhance the regeneration of osteoblast for treating arthritis.

In addition, the fermentation condition of the present invention is 4˜36 hours at 20˜50° C. preferably since the efficiency of fermentation is reduced at temperatures below 20° C. and above 50° C.

The present invention also provides an infusion method which comprises (1) increasing temperature slowly until it reaches 30˜75° C. during the saccharification process in order to reduce the time period and (2) having periods of pause for 30 minutes, first at 52° C. for protein synthesis and later at 65° C. for saccharification.

The present invention further provides herbal extract compositions itself for preventing and treating arthritis, which are prepared by using the above process.

The herbal extract compositions of the present invention are composed of Achyranthis root extract, Atractylodes japonica root extract and the synergistic mixture of the extracts. The herbal extracts of the present invention can be extracted separately or together and contain fermented products prepared by using the above process for pharmaceutical purposes.

Depending on the desired uses of the herbal extract compositions according to the present invention, one or more commonly used components such as vehicle can be added. Conventionally, the composition can be provided as the main pharmacologically active components in an oral dosage form including, but not limited to, tablets, capsules, caplets, syrup, liquid solutions, suspensions or powders, lozenges, micronized particles and osmotic delivery systems; or in a parenteral dosage form including unit administration or several times administration. Additionally, the herbal extract compositions comprise pharmaceutically acceptable components including solid carriers, liquid carriers, preservative agents, sweeteners, flavoring agents, coloring agents and combination thereof. The composition of the present invention described in Examples is prepared as the pharmaceutically active gel or soft extract form.

The dosage of the herbal compositions of the invention will vary, depending on factors such as severity of the arthritis, age, sex, physical condition, administration period, administration method, discharge ratio and body weight of the patient, diet, etc.

All kinds of arthritis can be within the scope of the present invention. Especially, contuse arthritis, rheumatoid arthritis, anaplastia arthritis, gouty arthritis, suppurative arthritis containing bacterial pus, tuberculous arthritis and the like are included.

The herbal compositions of the present invention have pharmaceutical effects for treating an autoimmune disease such as systemic lupus erythromatosus, amyloidosis and the like.

Further features and advantages of the present invention will appear hereinafter.

### BRIEF DESCRIPTION OF THE DRAWINGS

The above and other objects, features and other advantages of the present invention will be more clearly understood from the following detailed description taken in conjunction with the accompanying drawings, in which;

FIG. 1 represents the changes of paw volume in the process of time after injecting tuberculosis bacteria onto the experimental sample treated with the fermented herbal drug (4B) and in the control group.

FIG. 2 represents the changes of paw volume in the process of time after injecting tuberculosis bacteria onto the experimental sample treated with the non-fermented herbal drug (1B) and in the control group.

FIG. 3 represents the estimation of clinical arthritis index volume of arthritic mice with type II collagen in the process of time onto the experimental sample and in the control group.

FIG. 4 represents the estimation of arthritis incidence of arthritic mice with type II collagen in the process of time onto the experimental group and in the control group.

FIG. 5 represents the concentration variation of immunoglobulins in plasma obtained from the eyeballs of arthritic mice with type II collagen in the experimental group and in the control group.

FIG. 6 represents the concentration of intracellular TGF-β in ablated tissues of arthritic mice with type It collagen, which is analyzed by performing ELISA in the experimental group and in the control group.

FIG. 7 represents the concentration of intracellular INF-γ in ablated tissues of arthritic mice with type II collagen, which is analyzed by performing ELISA in the experimental group and in the control group.

FIG. 8 represents the concentration of intracellular TGF-β in ablated tissues of arthritic mice with type II collagen, which is analyzed by performing RT-PCR electrophoresis in the experimental group and in the control group.

FIG. 9 represents the concentration of intracellular IL-4 in ablated tissues of arthritic mice with type II collagen, which is analyzed by performing RT-PCR electrophoresis in the experimental sample and in the control sample.

FIG. 10 represents the concentration of intracellular TNF-α in ablated tissues of arthritic mice with type II collagen, which is analyzed by performing RT-PCR electrophoresis in the experimental group and in the control sample.

FIG. 11 represents the concentration of TNF-α of mouse macrophage which is stimulated with LPS and treated with herbal drugs prepared in Example 18 in the experimental group treated with hydrocortisone and the control group.

FIG. 12 represents the inhibition of collagenase activities by herbal drugs prepared in Example 18.

### DETAILED DESCRIPTION OF THE PREFERRED EMBODIMENTS

Achyranthes fauriei LEVEILLE et VANIOT or Achyranthes bidentata (soemureup) is a perennial plant in the Amaranthaceae family which grows to about 90 cm. Its root (Achyranthis Bidentatae Radix) contains saponin which can turn into oleanolic acid and glucuronic acid by hydrolysis, archyrantin (C₆H₁₁O₂N.H₂O) an alkaloid easily soluble in water and other alkaloids and large amount of mucus (viscous liquid). In its water extract, minerals are about 8% among which potassium salt is 24.5%. Amino acids including citosterol, stigmasterol, aspartic acid and multivalent basic acids including succinic acid and so on are also its components. In addition, it proves antispasmodic effect, urination, anti-allergic effect and so on as the pharmacological action (Yook, C. S. et al., Hyundai Herb Pharmacology, Hakchangsa, 24-128, 1993).

A medical decoction of Achyranthis roots (oosle) has been utilized in the oriental treatment for cleaning the blood, urination drug, emmenagogue. Also, it has been classified and administered as a haematopoietica in the oriental medicine.

The above Achyranthis root adopted in the present invention is a domestic natural herb, Achyranthes japonica (MIQ.) and a Chinese herb, Achyranthes bidentata Blume (hoeoosle) and both are purchased. The herb roots have been utilized to treat women's diseases such as puerperium inertia uteri, metremia induced by various causes, epimenorrhagia, which are described in reference documents.

Atractylodis Rhizoma (changchul) is a root stem of Atractylodes japonica KOIDZ (sapzoo) which is a perennial plant in the family Compositae and the genus Atractyloides and grows to about 80 cm. The above herbs grow wild in mountainous places and are lifted in spring or autumn, washed with water, trimmed on root remainders and dried in the sun.

Atractylodes japonica KOIDZ. roots (changchul) contains about 1.5% of essential oil and can react with carotin, inulin, gum and alkaloid. In addition, its [symbol] can react saponin and cumarine. The main component of the essential oil is atractylone (C₁₅H₂₀O, melting point 38° C., about 20% content), a sesquiterpene and also includes purpurale, β-eudesmol and hinesol. Referring to other documents, atractylol, isovalensic acid ester, atractylakalium and the like can be included. Besides, Atractylodes Koreana Kitam root (changchul) is reported to contain atractylodin, acetylatractylodinol, atractylodinol and elemole in small amounts as main components of essential oil. Among these components of Atractylodes japonica, atractylone is scarce and absent.

Referring to the description and drawings of ancient oriental documents, the root of Atractylodes Koreana Kitam is more close to Achyranthis roots. Achyranthis root is sweet, pungent and warm and has a effect upon ataralgesia, hematopoiesis and urination. Besides, it plays an important role such as small urination by kidney malfunction, gastroenteritis, edema, dizziness, whole body sickness, urination, sweating and so on in order to prescribe in the oriental treatment. On the other hand, as a folk medicine Achyranthis roots have been utilized to stop diarrhea and vomiting and to treat cacochymia, diabetes, cough and gout. Thus, it has been known to aid longevity if eating continuously.

The present inventors have confirmed that herbal extracts of Achyranthis roots and Atractylodes japonica roots and their mixture can used to make medicinal compositions. Concretely, the herbal compositions are identified to have little poisonous action and side effects and to treat arthritis efficiently and further their fermented products have examined to have better pharmaceutical efficacy. Therefore, we have developed new herbal extract-based pharmaceutical compositions which enhance the efficacies for treating above diseases in the present invention.

The present invention provides the herbal extract-based pharmaceutical compositions which is prepared by the process comprising the steps of; (1) extracting Achyranthis roots and Atractylodes japonica roots with water or alcohol respectively or together and (2) fermenting the herbal extracts and the like. The fermented products of the present invention have little side effects and high efficacies for preventing and treating arthritis. Hence, the herbal composition can be an excellent herbal component for developing pharmaceutical drugs and can be utilized to displace a conventional chemotherapeutic agent.

The present inventors have investigated to discover herbal components for treating arthritis derived from natural plants since the herbal extract based pharmaceutical composition has little side effects relatively. Presently, most of arthritis drugs are chemotherapeutic agents and has unfavorable side effects even although the severity depends on individual and the like. It is necessary to overcome the problems as follows; side effects preventing sustained uses of drug, lack of anti-inflammation and low efficacy against already generated arthritis. Hence, we have confirmed that extracts of Achyranthis Radix roots and Atractylodes japonica roots have pharmaceutical actions for treating rheumatoid arthritis in animal experiments etc. and prepared the herbal based pharmaceutical compositions. In addition, the fermented composition has been identified to be more effective for the treatment. Then the combination condition, the extraction protocol and the fermentation procedure are established by using dry herbs or natural raw herbs in order to complete the present invention.

The present invention relates to herbal extract compositions of Achyranthis roots and Atractylodes japonica roots for pharmaceutical purposes, their fermented products and the processes for the preparation the same. Preferably, in the fermentation process, cooked rice, starch and the like can be utilized, fermented with malt, yeast and the like and then evaporated for removing solvent. Particularly, the present invention relates to concentrated herbal extracts, their pharmaceutical compositions for preventing and treating arthritis such as conc. elixirs or pills, suspensions, capsules and so on and processes for preparation the same.

In detail, the present invention comprises concentrated extracts of Achyranthis roots (oosle), Atractylodes japonica roots (changchul) which can be extracted with low grade alcohol containing carbon number 1˜4, then exclude the remaining solvent and further can be applied for the fermentation procedure.

In order to examine the pharmaceutical efficacy for preventing and treating arthritis, herbal extract compositions of Achyranthis roots and Atractylodes japonica roots and their fermented products have administered into experimental rats. Concretely, the mice cells are collected in the drug treated group and the control group respectively and used to estimate clinical indices for the pharmaceutical analysis. The concentrations of cytokines related with arthritis and inflammation are measured for the analysis and in vitro experiment are performed by boosting mouse macrophages with LPS.

In the present invention, Chinese Achranthis bidentata BL in dried state or domestic Achyranthis japonica (MIQ.) NAKAI in natural and raw state is utilized as an Achyranthis root and then mixed with Atractylodes japonica root for preparing pharmaceutical herbal compositions. The herbal mixture is extracted with a solvent and fermented by using malt, yeast or so on. The fermented product is examined to measure the efficacy for preventing and treating arthritis and excellent results are obtained in the animal experiments and in the cell histological analysis.

The extracts of Achyranthis roots and Atractylodes japonica roots can be prepared with the herbal extraction procedure as follows.

Achyranthis roots and Atractylodes japonica roots are mixed with an extraction solvent and heated at 50˜120° C. for 5˜24 hours. The extracted solution is cooled at 40˜60° C. and filtra obtain supernatant or applied for the next fermentation procedure directly. The above procedures of extraction and filtration are repeated more than once and then collects supernatant and evaporates its solvent to concentrate. Achyranthis roots and Atractylodes japonica roots can be extracted respectively and then combined or extracted together, but the co-extraction protocol is more convenient to be performed. When raising temperature, the extract is heated in an oil bath at 120° C. or uses the steam distillation method. Preferably, it is warmed in a water bath at 80˜100° C. In case that distilled water is used as a solvent, the amount of dried Atractylodes japonica root is 12-fold optimally in the experimental range of 6˜16 fold.

Achyranthis roots and Atractylodes japonica roots are mixed with an extraction solvent, immersed in a cool bath at normal temperature or 4° C. for 5˜7 days and filtrated to obtain the supernatant. The above procedures of extraction and filtration are repeated more than once and then collects supernatant and evaporated its solvent to concentrate.

In two processes for the preparation demonstrated above, Achyranthis roots and Atractylodes japonica roots can be reduced to powder and as an extraction solvent, pure water or alcohol solution such as 20˜50% of ethanol, 50˜100% of methanol and the like can be utilized. But organic solvent is removed completely to apply for the next fermentation process.

The process for next fermentation of the present invention can be accomplished as follows.

The herb extract can be fermented preferably at 20˜50° C. for 4˜36 hours by adding malt, yeast and the like since the efficiency of fermentation is reduced at temperature below 20° C. and above 50° C. At that time, the herbal extract composition is prepared by using the method described above and uses filtrated supernatant or is fermented directly and then filtrated. In case that the herbal extract is fermented without the filtration process, the more fermented product can be obtained about 5% than in case that it is filtrated before the fermentation.

The present invention also provides an infusion method for rapid fermentation which increases temperature slowly from 30° C. and allows pause periods first at 52° C. for protein synthesis and later at 65° C. for saccharification for 30 minutes respectively. As a result, the final temperature for the fermentation reaches 75° C. in 2 hours 45 minutes and the filtration in a high temperature can reduce the operating time.

In addition, the fermentation step can be performed naturally or by adding malt, yeast, grape, wine enzyme source or other microorganisms. Since herbal compositions of the present invention are fermented, drug efficacy, taste, flavor and the like are improved favorably and especially the above fermentation makes a pharmaceutical form of drug sustained and stored for long time. Besides, the fermentation step can be accomplished by the process that cooked rice is fermented with malt, yeast and the like, filtrated to obtain supernatant and then fermented by adding the herb extract of the present invention.

In the utilization of malt, starch contained in malts can be saccharified with its enzymes and change to suitable extracts for the fermentation. At that time, by-substrate is used for replenishing the malt starch until reaching 50% amount of malt. This is economical due to cheap price of by-substrate and gives a lot of additional effects such as flavour promotion, reduced turbidity and so on. Besides, this improves the fermentation efficiency such as reduced fermentation period, increased fermented products and so on. As a by-substrate, refined starches such as corn, rice, wheat, papain derived from caprica papaya, kiwi, pear and the like can be utilized and glucose syrup, such as corn syrup saccharifying these starches with acids or enzymes, can be also adopted.

Generally, malted wheat (koji) source can be selected among wheat, rice coated with wheat powder, mung beans, glutinous rice, barley and the like. In addition, yeast source can be selected among air, cocklebur, barley straw, paddy straw, mulberry tree, mugwort, lettuce, lotus. flower, water pepper leaf, pine tree leaf and the like. Besides, physical states of the above seeds can be the roasted, the steamed, the slightly steamed or raw wheat and the threshed ratio can be in the range of 0˜12 percentage. By-substrate can be selected among mulberry tree leaf, mugwort, decocted soup, peach seed powder, melon powder and the like. Then, co-enzyme agents are added for the preparation, which improves the saccharification activity by cultivating conventional malted wheat, such as major bacteria Rhyzopus, Usami, Oryzae and so on but maintains complex taste of natural malts.

The preferable method of processing the compositions of the invention for ingestion is to package the powdered herbal mixture into gelatin capsules (preferably hard gelatin) of a size preferably of the order of zero or double zero. Such capsules would then contain about 300˜600 mg of the powdered herbal mixture per capsule. It has been found that hard gelatin capsules represent the most efficient, economical form of packaging the edible composition for ingestion.

The dosage of the herbal compositions of the invention to be ingested will vary, depending on factors such as severity of the arthritis, age, physical condition and body weight of the patient, diet, etc. As a general guide, it is expected that patients with a body weight in the range of 60˜90 kg would ingest about 1,000˜5,000 mg/day of the herbal compositions (corresponding to 2˜12 zero or double zero size hard gelatin capsules per day). Typically, a human patient would ingest about 40 mg of the composition per kg of body weight. It is to be understood that these dosage levels are only general guides and the proper dosage level for individual patients may vary considerably depending on the factors indicated above. However, one benefit of the edible compositions of the present invention is that the dosage is not “critical” as is the case with administration of synthetic pharmaceutical medications such as those mentioned above. Since the edible compositions of the present invention are holistic in nature and represent dietary supplements in their own right, “overdosing” is not a problem.

The individual patient with a particular body weight and life style may readily determine the proper dosage by starting out with the general dosage level set forth above and adjust the dosage as necessary to alleviate the arthritis.

### BEST MODE FOR CARRYING OUT THE INVENTION

### EXAMPLES

Practical and presently preferred embodiments of the present invention are illustrated in the following Examples.

However, it will be appreciated that those skilled in the art, on consideration of this disclosure, may make modifications and improvements within the spirit and scope of the present invention. Unless otherwise indicated, all amounts and parts are on a weight basis, especially all % signs indicate weight %.

### Example 1

85 g of wild Achyranthis roots (water content 50%) was washed well and dried in the shade and 85 g of dried Atractylodes japonica roots were cut into small pieces. Then the herbs were put into 2 L round flask and 500 mL of water was added. After perfusion refrigerator was equipped, the herb mixture was steam-extracted for 10 hours at 120° C. The extracted solution was cooled down to about 75° C. and filtrated at high temperature. The obtained supernatant was concentrated through the process of solvent evaporation and was freeze-dried so as to obtain 55 g of pills (the preparation of 1B sample).

### Example 2

85 g of wild Achyranthis roots (water content 50%) was washed well and dried in the shade and 85 g of dried Atractylodes japonica roots were cut into small pieces. Then the herbs were put into 2 L round flask and 500 mL of water was added. After perfusion refrigerator was equipped, the herb mixture was steam-extracted for 10 hours at 120° C. The extracted solution was cooled down to about 50° C. was put together with 60 g of malt and 85 g of cooked rice and was fermented at 45° C. for 12 hours. The fermented product was filtrated and the obtained supernatant was concentrated through evaporation process so as to produce liquid extracts. Then the extracts were freeze-dried to obtain 64 g of pills (the preparation of 4B sample).

### Example 3

40 g of wild Achyranthis roots (water content 50%) was washed well and dried in the shade and 40 g of dried Atractylodes japonica roots were cut into small pieces. Then the herbs were put into 5 L round flask and 1280 mL of distilled water was added. After perfusion refrigerator was equipped, the herb mixture was extracted in water bath for 10 hours at 80˜100° C. The extracted solution cooled about 50° C. was put together with 12 g of malt and 32 g of cooked rice and was fermented at normal temperature (warm place of about 30° C.) for 36 hours. When the cooked rice floated onto the liquid surface, the fermentation process was completed. The resulting extracted product was filtrated as the temperature was raised to 75° C. and the obtained supernatant was concentrated through solvent evaporation so as to produce 85 ml of liquid extracts (the sample of Experimental Example 2).

### Example 4

20 g of dried Achyranthis roots (water content 0.7%) and 40 g of dried Atractylodes japonica roots were cut into small pieces and put into 2 L round flask and 480 mL of water was added. After perfusion refrigerator was equipped, the herb mixture was extracted in oil bath for 10 hours at 120° C. The extracted solution was cooled to about 75° C. and filtrated at high temperature. Then the obtained supernatant was concentrated through solvent evaporation so as to produce 31.5 g of solid powder extracts. In comparison with the result of Example 1, about 20% more crude extract was obtained.

### Example 5˜Example 7

40 g of wild Achyranthis roots (water content 50%) was washed well and dried in the shade and 40 g of dried Atractylodes japonica roots were cut into small pieces. Then the herbs were put into 2 L round flask and distilled water was added. After perfusion refrigerator was equipped, the herb mixture was extracted in oil bath for 10 hours at 120° C. The extracted solution was cooled to about 50° C. and 7 g of malt was added to the extracted solution in order to be fermented at 45° C. for 12 hours. Then the resulting extract was filtrated and concentrated through solvent evaporation so as to produce solid powder.

### Example 8

20 g of dried Achyranthis roots (water content 50%) and 40 g of dried Atractylodes japonica roots were cut into small pieces. Then the herbs were put into 2 L round flask and 480 ml of water was added. After perfusion refrigerator was equipped, the herb mixture was extracted in oil bath for 10 hours at 120° C. 7 g of malt and 3 g of starch were added to the extracted solution which was cooled to about 50° C. in order to be fermented at 45° C. for 6 hours. Then the resulting extract was filtrated and concentrated through solvent evaporation so as to produce 36.8 g of solid powder.

### Example 9

20 g of dried Achyranthis roots (water content 0.7%) and 40 g of dried Atractylodes japonica roots were cut into small pieces. Then the herbs were put into 2 L round flask and 480 mL of water was added. After perfusion refrigerator was equipped, the herb mixture was extracted in oil bath for 10 hours at 120° C. 7 g of malt and 3 g of starch were added to the extracted solution which was cooled to about 75° C. in order to be fermented at 45° C. for 6 hours. Then the resulting extract was filtrated and concentrated through solvent evaporation so as to produce 34.6 g of solid powder.

### Example 10

20 g of dried Achyranthis roots (water content 0.7%) and 40 g of dried Atractylodes japonica roots were cut into small pieces. Then the herbs were put into 2 L round flask and 480 mL of water was added. After perfusion refrigerator was equipped, the herb mixture was extracted in oil bath for 10 hours at 120° C. 7 g of malt and 3 g of starch were added to the extracted solution which was cooled to about 75° C. Then the solution was heated slowly until the temperature reaches 30˜75° C. and there were two 30 minutes pauses at 52° C. and at 65° C. After 2 hours 30 minutes, when the temperature of solution finally reached 75° C., the solution was left as it was for 15 minutes and filtrated for 6 hours. Then the above supernatants of herb extract were collected and fermented for 6 hours. Then the resulting extract was filtrated and concentrated through solvent evaporation so as to produce 34.1 g of solid powder.

### Example 11˜Example 14

20 g of dried Achyranthis roots and 40 g of dried Atractylodes japonica roots were cut into small pieces. The herbs were put into 2 L round flask and 480 mL of 100% methanol was added as a solvent. The mixture was extracted in water bath at 80° C. for 10 hours. The resulting herbs were extracted and filtrated for obtaining supernatant. Then the herb extract was concentrated by evaporating solvent and turned into solid powder. This was fermented with 80 ml of distilled water for 6 hours.

### Example 15

20 g of dried Achyranthis roots and 40 g of dried Atractylodes japonica roots were cut into small pieces and were put into 2 L round flask. The herb was immersed in 480 mL of 80% methanol (methanol: water=4:1 v/v) solvent at 4° C. for 7 days. The resulting herb extracts were filtrated and the obtained supernatant was concentrated through solvent evaporation. When the remaining methanol was completely removed, 34.6 g of solid powder was produced.

### Example 16

400 mL of distilled water, 100 g of malt (dried barley sprouts) and 100 g of fermentation by-substrate, starch such as cooked rice or wheat, corn, papain and so on were mixed. Then the fermented products were prepared by using the extraction method of increasing temperature to 30˜75° C. After being filtrated at high temperature of 75° C., clear supernatant was used in the present invention.

### Example 17

Raw material wheat was selected, washed and dried sufficiently and then smashed and kneaded with 20˜25% of water sprinkled. The standard amount of this mixture was put into a yeast frame and pressed into a shape. Then wheat powder mixed with germ bacteria (Aspergillus oryzae derived from Changmo Castle with denser green color; Aspergillus niger mut. Kawachii and the like can be utilized) was smeared on the above shaped mixture as a surface injection. The resulting paste was spread on a shelf in the grain germ room, in between two layers of the sterilized straw cushions and incubated. Then ventilating and controlling the temperature below 35° C. dried it. At the same time, the mixture was turned over every day or every other day. As a result, spores with yellowish green color appeared after 4˜5 days of incubation and the surface of germ grains started to dry up when the temperature of incubation was over 40° C. Then the mixture block was left as it was as the above temperature fell and delivered from the grain room after 8˜10 days. After the maturation process of 7 days, the above block was stored in the drying room in order to be used.

### Example 18

The amounts of dried Achyranthis roots (oosle) and Atractylodes japonica roots (changchul) were measured exactly and they were circulation-extracted for 12 hours with methanol which was 8 times (v/wt) as much as Atractylodes japonica root Then the extracted product was filtrated and concentrated by reducing pressure and removing solvent In order to ferment the resulting extract, each batch of the extracted solution was mixed with the fermented product prepared in Example 16 and Example 17 and incubated at 45° C. for 12 hours.

### Example 19

The extract powder of the present invention was prepared with the same process of Example 8 and pharmaceutically acceptable carriers were added so as to obtain micronized particles and suspensions. Then the above extract compositions were made to be hard capsules.

### Example 20

The extract powder of the present invention was prepared with the same process of Example 8 and a small amount of ethanol for an alcoholic beverage and distilled water were added so as to make soft capsules.

### Experimental Example 1

Heat-treated Mycobacterium butyricum was purchased from Disco company (Detroit, Mich.) and male Wistar Lewis Rat was bought from Charles River Japan Inc. The body weights of rats were adjusted to about 250 g after purchasing (starting from the initial weight of about 180 g). Reagent like incomplete Freund adjuvant was purchased from Sigma Company.

In case of AA (acute arthritis), Mycobacterium butyricum (MB) mixed with Freund adjuvant was made to suspension (5 mg/ml) and injected once onto the hypodermic layer of the right sole in Wister Lewis Rat, 100 μl per each animal, which was considered as a control group. On the other hand, the standard group was injected with only Freund adjuvant excluding MB in the same amount subcutaneously. The volumes of both paws were measured in every 3 or 4 day by using the water displacement method, which continued for about 28 days. The pharmaceutical composition of the present invention was administered by 2 g/kg on the basis of solid form weight and each experimental group had about 6˜7 members (Turull and Queralt, 2000; Immuno. Pharmacol., 46 (2000) 71-77).

In each group of the figure, designation and the number of animals were as follows.

Control group: 6 animals

IB, 4B group: drug-treated experimental group(respective 7 animals)

Standard group: 6 animals

Paw volume change was defined as the volume differences between the control group and the standard group and between the experimental group and the standard group. In the experimental group, the inhibition effect of the administered drug in AA was defined as the following formular (Badger, et al., Journal of Pharmacology and Experimental Therapeutics, 291 (1999), 1380˜1386). In this experiment, 1B indicated a non-fermented herbal extract (Example 1) and 4B meant a fermented herbal extract (Example 2).

The inhibition effect (%)={1−[experimental group−standard group]/[control group−standard group]}×100

The statistic analysis was accomplished to calculate p values by using Student's t test and the values of the paw volume in the experimental group and the control group and to compare. In case that p value is below 0.05, it was considered to be significant statistically.

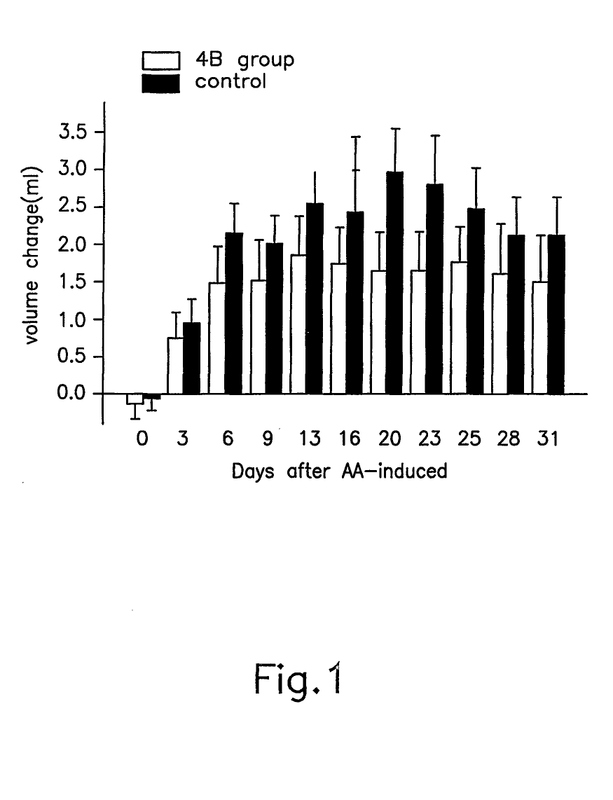

FIG. 1 summarized the volume changes of paws in the process of time after the injection of tuberculosis bacteria (TB). As shown in the figure, the control group indicated the initial change in the third day and the volume increased to 2.6 times of the first day. This increasing pattern was maintained for 16 days and again an additional increase was observed in the 20th day, which showed 3.7-fold increase of the initial volume. Then it turned to the decreasing pattern and was 2.9 times of the initial value in about 31 days. When administered 4B, the general pattern was similar to that of the control group except that the 4B group sustained or reduced third volume until the experiment finished. Concretely, the paw volume of 4B group was reduced to 55% of the control group in the 20th day (p<0.001), which meant this group had a decreasing pattern compared with the control group all through the process. On the other hand, the standard group showed the maximum volume in the initial 3 days (1.8 times larger than the first day) and then the paw volume reduced gradually so as to recover the initial value or its slightly increased value.

Besides, the average weights of the experimental group, the control group and the standard group were almost the same value of about 270 g. But about 20 days after AA was induced, the weight was reduced by 25 g in 4B, 1B and the control group respectively. On the other hand, the weight of the standard group increased by about 60 g and the left paw volumes of both groups were not different from the initial value.

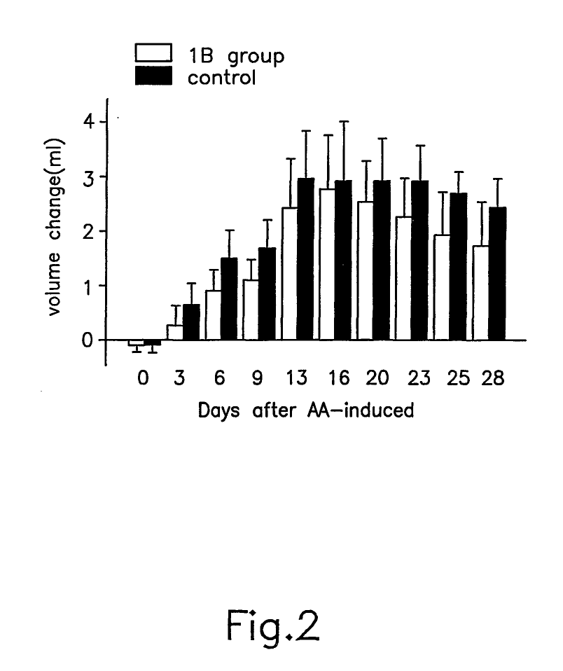

As illustrated in FIG. 2, when 1B was administered in the control group, the volume increased at first in the similar pattern and after some time (in about 2 weeks) increased again remarkably. Such a tendency was clearly different from that shown in FIG. 1 of 4B injection. 1B was proved to reduce edema effectively when compared with the control group. And 4B was identified to have more excellent effect in reducing edema when compared with the control group.

TABLE 1
The difference of right paw volumes and significances (p)
in 4B, 1B compared with the control group.

| days | | | | | |
|---|---|---|---|---|---|
| (4B) | difference (mL) | p value | Days (1B) | difference (mL) | p value |
| 0 | 0.08 | 0.36 | 0 | 0.01 | 0.82 |
| 3 | 0.20 | 0.063 | 3 | 0.38 | 0.03 |
| 6 | 0.65 | 0.025 | 5 | 0.6 | 0.03 |
| 9 | 0.46 | 0.097 | 8 | 0.6 | 0.07 |
| 13 | 0.68 | 0.010 | 14 | 0.55 | 0.28 |
| 16 | 0.69 | 0.149 | 17 | 0.55 | 0.2 |
| 20 | 1.31 | 0.001 | 19 | 0.73 | 0.05 |
| 23 | 1.14 | 0.004 | 23 | 0.89 | 0.02 |
| 25 | 0.71 | 0.027 | 25 | 0.77 | 0.06 |
| 28 | 0.51 | 0.129 | 28 | 0.72 | 0.09 |
| 31 | 0.62 | 0.059 | | | |

(Bold characters indicated statistically significant values (p < 0.05))

Table 1 summarized the differences between the edema which was measured in the right paws when administered 4B and 1B and that of the control group. As illustrated in FIG. 1, the edema was improved in 4B injection favorably and remarkably during 2˜4 weeks compared with the control group but in 1B injection, the significance was relatively slight although it sustained some efficacy.

TABLE 2
Edema inhibition of 4B and 1B injection

| days (4B) | inhibition (%) | days (1B) | inhibition (%) |
|---|---|---|---|
| 0 | | 0 | |
| 3 | 20.4 | 3 | 58.5 |
| 6 | 30.1 | 5 | 34.1 |
| 9 | 22.9 | 8 | 24.5 |
| 13 | 26.6 | 14 | 18.2 |
| 16 | 28.2 | 17 | 5.75 |
| 20 | 44.1 | 19 | 22.0 |
| 23 | 40.6 | 23 | 27.7 |
| 25 | 28.4 | 25 | 28.0 |
| 28 | 23.8 | 28 | 29.0 |
| 31 | 29.0 | | |

(Bold characters indicated statistically significant values (p <0.05))

Table 2 showed the converted result of the above inhibition effects and the most excellent effect was with the 4B administration. Concretely, the 4B injection induced the reduction of MB edema by 40˜45% and the 1B injection brought about 25% of the reduction. Consequently, in both Achyranthis roots (oosle) and Atractylodes japonica roots (changchul) the fermented product (4B) had higher effects for treating arthritis than the non-fermented product (1B).

### Experimental Example 2

Estimation of Clinical Arthritic Index

46 experimental animals of DBA/1 mice derived from Jackson Lab. USA were obtained from Korea Institute of Science and Technology (KIST) and adjusted to have 21.5 g of the average weight. Then they were stabilized for 2 weeks, separated into the control group and the experimental group and injected onto mouse tail with type II collagen for the primary immunization so as to examine type II collagen-induced mouse arthritis test After 2 weeks, they were boosted again by injecting on the left sole similarly and then 20 animals among the experimental group were administered orally for 2 weeks, 7 times with 1 trials per 2 days, in 0.6 g/kg weight (122.5 μl/animal) of solid powder prepared in Example 3 (a fermented herbal extract composition). Arthritis incidence and arthritis induction ratio of the experimental group (drug treated group, tolerance group) were examined by using clinical arthritic index, hereinafter “AI”) and comparing those of the comparative group (CIA group). In order to estimate the efficacy of herbal extract compositions for treating arthritis, 4 mice of the control group were boosted and victimized in 2 days and then analyzed by using cytohistologically. In addition, the examination standard was obtained and after 5 weeks since the experiment starts and the drug treatment was completed, 4 mice were victimized secondarily and respectively in the experimental group and the comparative group and their cell tissues were ablated and cultivated so as to measure the treatment efficacy for anti-inflammation and arthritis by performing ELISA, RT-PCR method and the like.

In order to accomplish the immunization, CFA (complete Freund's adjuvant) reagent was Arthrogen-CIA® (5 ml/vial Lot. 112700) of Chondrex company and type II collagen was Bovine native C II of Sigma company and mixed with CFA so as to be 1:1 ratio and to be injected with 100 μl per 1 animal into the mice tails. When boosted, IFA (incomplete Freund's adjuvant) purchased from Difco company was mixed with Bovine native C II with 1:1 ratio and also injected with 100 μl per 1 animal into the left foot pad of mice. In order to measure the degree of the arthritis induction, clinical arthritis index was determined by observing the arthritis induction of remaining 3 feet excluding the left foot. Concretely, according to the edema 0˜4 scores were allotted (score 0: edema not appears; 1: rubor, red swelling; 2: rubor and edema appearance; 3: arthritis induction, swelling like the edema of the left foot pad; 4: severe inflammation, crack, difficulty of standing with feet) and summed up. The incidence was depicted with % of the number of mice which had remarkable edema appearance (more than 3 score of AI estimation).

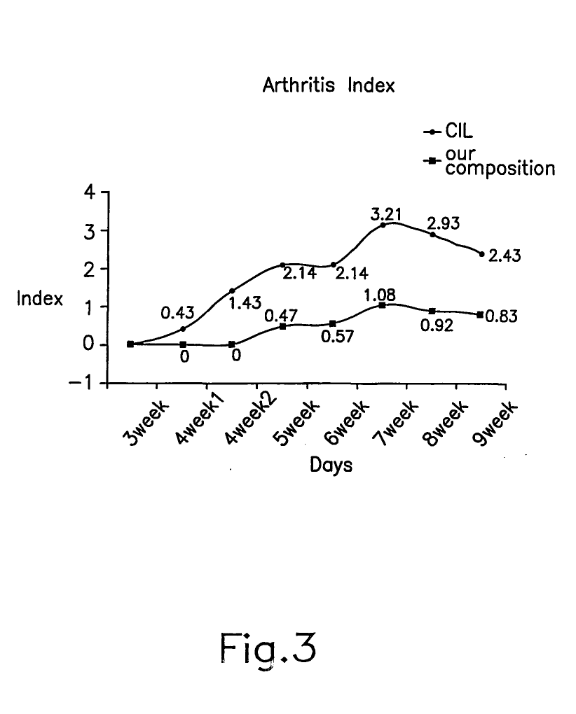

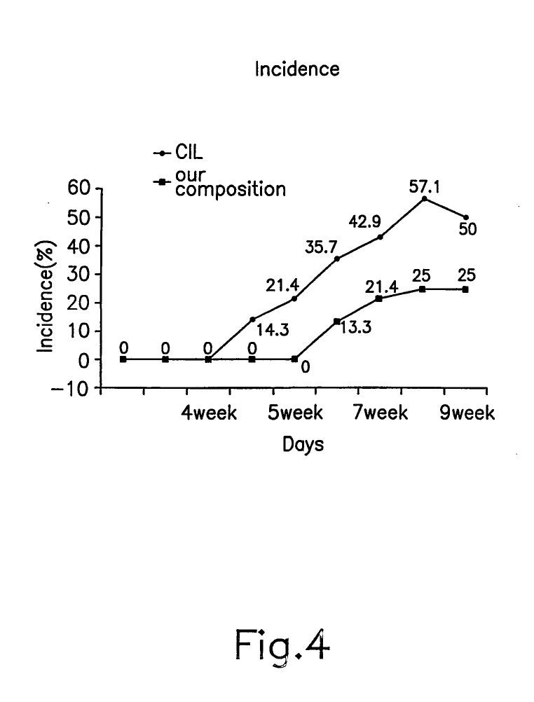

Table 3 and Table 4 summarized the estimation of AI and incidence which were obtained by performing clinical estimation in 3 weeks since giving the experimental animals the herbal extract and were illustrated in FIG. 3 and FIG. 4. Then the herbal drug of the present invention was treated for 4 weeks and after 8 weeks arthritis induction increased to the maximum point of 57.1% in the control group (CIA group), became sure to have more than 3 score of average AI estimation and then decreased. In drug treated group, the incidence was shown to increase after 6 weeks of drug administration but arthritis induction and the like were not observed during the treatment period. Therefore, the drug efficacy for preventing and treating arthritis was confirmed clearly. And the AI estimation analysis was shown that AI value increased primarily during 5 weeks and increased secondarily in 7 weeks and this was the same pattern described in Experimental Example 1 definitely. Then the progress was recorded that the maximum point of AI estimation was below average 1 score in the drug treated group and the treatment efficacy was 66.4% of remarkable results in CIA group.

TABLE 3
Clinical evaluation chart of CIA group
Arthritic index

| Time | I group | | | II group | | | III group | | | | | IV group | | | |
|---|---|---|---|---|---|---|---|---|---|---|---|---|---|---|---|
| (week) | 1 | 2 | 3 | 4 | 5 | 6 | 7 | 8 | 9 | 10 | 11 | 12 | 13 | 14 | Average |
| 3 | 0 | 0 | 0 | 0 | 0 | 0 | 0 | 0 | 0 | 0 | 0 | 0 | 0 | 0 | 0 |
| 4-1 | 0 | 0 | 0 | 0 | 0 | 0 | 0 | 1 | 1 | 0 | 0 | 1 | 1 | 2 | 0.43 |
| 4-2 | 0 | 2 | 1 | 3 | 1 | 2 | 1 | 0 | 0 | 2 | 0 | 2 | 4 | 2 | 1.43 |
| 5 | 0 | 2 | 1 | 3 | 1 | 1 | 1 | 2 | 3 | 3 | 0 | 6 | 5 | 2 | 2.14 |
| 6 | 0 | 2 | 0 | 2 | 0 | 1 | 1 | 6 | 3 | 2 | 1 | 4 | 5 | 3 | 2.14 |
| 7 | 3 | 2 | 1 | 1 | 3 | 3 | 5 | 7 | 1 | 4 | 3 | 4 | 5 | 3 | 3.21 |
| 8 | 3 | 1 | 2 | 1 | 3 | 2 | 5 | 6 | 1 | 4 | 2 | 3 | 5 | 3 | 2.93 |
| 9 | 2 | 1 | 1 | 2 | 2 | 2 | 4 | 5 | 1 | 3 | 2 | 2 | 4 | 3 | 2.43 |

Incidence

| Time | I group | | | II group | | | III group | | | | | IV group | | | |
|---|---|---|---|---|---|---|---|---|---|---|---|---|---|---|---|
| (week) | 1 | 2 | 3 | 4 | 5 | 6 | 7 | 8 | 9 | 10 | 11 | 12 | 13 | 14 | average |
| 3 | − | − | − | − | − | − | − | − | − | − | − | − | − | − | 0 |
| 4-1 | − | − | − | − | − | − | − | − | − | − | − | − | − | − | 0 |
| 4-2 | − | − | − | − | − | − | − | − | − | − | − | + | + | − | 14.3 |
| 5 | − | − | − | − | − | − | − | − | + | − | − | + | + | − | 21.4 |
| 6 | − | − | − | − | − | − | − | + | − | + | + | + | + | − | 35.7 |
| 7 | − | − | − | − | − | − | + | + | − | + | + | + | + | − | 42.9 |
| 8 | − | − | + | − | − | − | + | + | − | + | + | + | + | + | 57.1 |
| 9 | − | − | − | − | − | − | + | + | − | + | + | + | + | + | 50 |

TABLE 4
Clinical evaluation chart of drug treated group
Incidence

| | I group | | | | II group | | | | | III group | | | IV group | | | | |
|---|---|---|---|---|---|---|---|---|---|---|---|---|---|---|---|---|---|
| time | 1 | 2 | 3 | 4 | 5 | 6 | 7 | 8 | 9 | 10 | 11 | 12 | 13 | 14 | 15 | 16 | average |
| 3 | 0 | 0 | 0 | 0 | 0 | 0 | 0 | 0 | 0 | 0 | 0 | 0 | 0 | 0 | 0 | 0 | 0/16 = 0 |
| 4-1 | 0 | 0 | 0 | 0 | 0 | 0 | 0 | 0 | 0 | 0 | 0 | 0 | 0 | 0 | 0 | (2) | 0/15 = 0 |
| 4-2 | 0 | 0 | 0 | 0 | 0 | 0 | 0 | 0 | 0 | 0 | 0 | 0 | 0 | 0 | 0 | 0 | 0/16 = 0 |
| 5 | 0 | 0 | 1 | 0 | 0 | 0 | 1 | 1 | 0 | 0 | 1 | 1 | 1 | (1) | 0 | 1 | 7/15 = 0.47 |
| 6 | 1 | 0 | | 1 | 0 | 0 | 0 | 0 | 0 | | 0 | 4 | 0 | 0 | 0 | 2 | 8/14 = 0.57 |
| 7 | 0 | 0 | | | 0 | | 1 | 1 | 2 | | 4 | 0 | 2 | 0 | 1 | 2 | 13/12 = 1.08 |
| 8 | 0 | 0 | | | 0 | | 1 | 1 | 2 | | 3 | 0 | 2 | 0 | 0 | 2 | 11/12 = 0.92 |
| 9 | 0 | 2 | | | 0 | | 1 | 0 | 2 | | 3 | 0 | 2 | 0 | 0 | 0 | 10/12 = 0.83 |

Incidence

| | I group | | | | II group | | | | | III group | | | IV group | | | | |
|---|---|---|---|---|---|---|---|---|---|---|---|---|---|---|---|---|---|
| time | 1 | 2 | 3 | 4 | 5 | 6 | 7 | 8 | 9 | 10 | 11 | 12 | 13 | 14 | 15 | 16 | Average |
| 3 | − | − | − | − | − | − | − | − | − | − | − | − | − | − | − | − | 0/16 = 0 |
| 4-1 | − | − | − | − | − | − | − | − | − | − | − | − | − | − | − | (−) | 0/15 = 0 |
| 4-2 | − | − | − | − | − | − | − | − | − | − | − | − | − | − | − | − | 0/16 = 0 |
| 5 | − | − | − | − | − | − | − | − | − | − | − | − | − | (−) | − | − | 0/15 = 0 |
| 6 | − | − | − | − | − | − | − | − | − | | − | + | − | − | − | + | 2/15 = 13.3 |
| 7 | − | − | | − | − | − | − | − | + | | + | − | − | − | − | + | 3/14 = 21.4 |
| 8 | − | − | | | − | | − | − | + | | + | − | − | − | − | + | 3/12 = 25.0 |
| 9 | − | + | | | − | | − | − | + | | + | − | − | − | − | − | 3/12 = 25.0 |

### Experimental Example 3

Histological Analysis of Arthritis Induced and Ablated Mouse Cell

In animal experiments of Experimental Example 2, mice were boosted in 2 weeks and 4 animals of the control group were victimized primarily before administering drugs. After drug injection was finished in 2 weeks, 4 mice were victimized respectively in the comparative group (CIA group) and the drug treated group. Then each region of tissue was ablated and cultivated by using ELISA and RT-PCR method so as to examine the intracellular inflammation and the concentration variation of cytokines related with immunization and anti-inflammation and arthritis treatment effects. In addition, the amount of immunoglobulins obtained from blood plasma of eyeball were measured by the antibody titration and total amounts of immune protein, IgG1, IgG2 were calculated so as to analyze drug activity toward T cell.

In the experimental mice, tissue cells were ablated from the region selected among dLN (drainic lymph node), mLN (mensenteric lymph node), spleen and PP (Peyer's patches). Then cell soup was prepared with RPMI 1640 medium/10% solution and centrifuged at 3,000 rpm, for 5 minutes to be seeded with 5×10⁵ cells/well of cell concentration. ELISA (enzyme linked immunosorbent assay) method was performed by using SPECTRA MAX 250 instrument in order to assay TGF-β (transforming growth factor-β) and INF-γ (interferone-γ). RT-PCR (reverse transcription-polymerase chain reaction) method was accomplished by using Perkin Elmer 9600 in order to measure the concentration differences of intracellular TNF-α (tumor necrosis factor-α), IL-4 (interleukin-4) and TGF-β in the CIA group and tolerance group and compare anti-inflammation and arthritis treatment.

Table 5 and Table 6 described the ELISA analysis process of TGF-β and INF-γ and as a blocking solution, PBS reagent of GIBCO BRL® which was prepared by mixing 9.6 g of Dulbecco's phosphate-buffered saline and distilled water to make 1 L volume and by adding 10 g of 1% BSA (TM-USB. Lot 106268) and 0.1 % of sodium acetate, was utilized. At that time, in order to measure TGF-β the antibodies of #244 (Catalog No. MAB 1835) and #19 (Lot. No. WS04) were purchased from R & D System company, USA and as a standard the antibodies of #17 (Cat No. 100-21) and #18 (Cat No. 80-1835) were bought from PEPRO Tech EC LTD, USA and Genzyme/Techne company were utilized considering the manuals of the catalogues.

TABLE 5
ELISA procedure/TGF-β

| | |
|---|---|
| 1 | Dilute #244 coated plate at 4° C. over night (1/15) −> (6 ul + 894 ul |
| | cc PBS) 3 ml −> 50 ul/well. |
| 2 | Washing condition: PBST 1X |
| 3 | Block the plate with blocking solution and incubate at RT for 1 hour. |
| 4 | Washing condition: PBST 1X |
| 5 | Incubate #17 standard sample at RT for 2 hours (for dilution: 2 ul + |
| | 238 ul blocking solution corresponding to 2 ug/ml −> 2000 pg). |
| 6 | Washing condition: PBST 1X |
| 7 | Detect #19 antibody and incubate at RT for 1 hour 30 minutes (for |
| | dilution: 1/150, 1.8 ul + 898.2 ul blocking solution −> 50 ul/well. |
| 8 | Washing condition: PBST 1X |
| 9 | Incubate secondary enzyme (Streptavidin-HRP ® R&D System |
| | company: dilution 1/200) in the shade for 30 minutes. |
| 10 | Washing condition: PBST 1X |
| 11 | Mix substrates A:B = 1:1 −> 50 ul/well dilution and incubate for 30 |
| | minutes ˜ 1 hour according to coloring. |
| 12 | Stop the reaction with 1 N HCl stopping solution. |
| 13 | Read at 450 nm by ELISA analyzer |

TABLE 6
ELISA procedure/INF-γ

| | |
|---|---|
| 1 | Dilute #352 coated plate at 4° C. over night (1/500) −> (3 μl + 2997 |
| | μl cc PBS) −> 50 μl/well cc PBS. |
| 2 | Washing condition PBST 1X |
| 3 | Block the plate with blocking solution (dilution: 50 μl/well) and |
| | incubate at RT for 1 hour. |
| 4 | Washing condition: PBST 1X |
| 5 | Incubate #344 standard sample at RT for 2 hours (for dilution: 1/100, |
| | 2.4 μl + 240 μl blocking solution −> 1/2). |
| 6 | Washing condition: PBST 1X |
| 7 | Detect #353 antibody and incubate at RT for 1 hour 30 minutes (for |
| | dilution: 1/250, 3.6 μl + 900 μl blocking solution). |
| 8 | Washing condition: PBST 1X |
| 9 | Incubate secondary enzyme (Streptavidin-HRP ® R&D System |
| | company: dilution 1/200 −> 15 μl + 2985 μl) in the shade for 30 |
| | minutes. |
| 10 | Washing condition: PBST 1X |
| 11 | Mix substrates A:B = 1:1 −> 50 μl/well dilution and incubate for 30 |
| | minutes ˜ 1 hour according to coloring. |
| 12 | Stop the reaction with 1 N HCl stopping solution. |
| 13 | Read at 450 nm by ELISA analyzer |

In order to perform ELISA analysis of INF-γ, as a standard the antibody reagents of #352 (Cat. No. MAB 785), #353 (Cat No. MAF485) and #344 (Cat. No. 485-MI) were purchased from R & D System company and utilized considering the manuals of the catalogues.

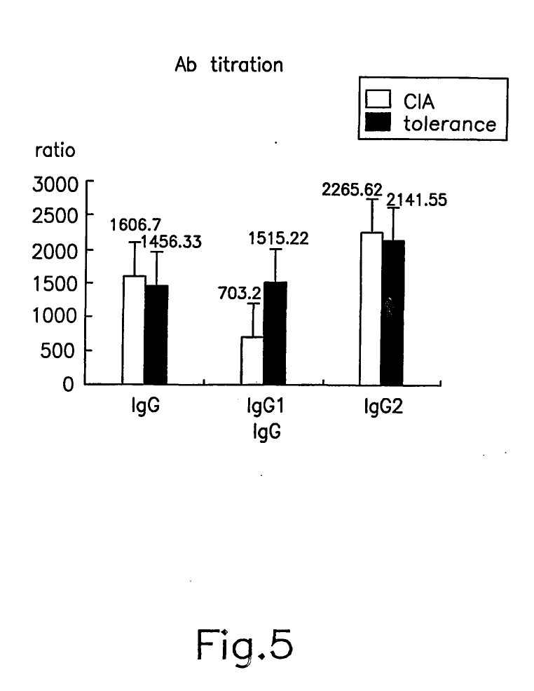

FIG. 5 represents the concentration variation of immunoglobulins in plasma obtained from mouse eyeball in the experimental sample and the standard sample of arthritis induced mice with type II collagen. 20 μg/ml concentration of native bovine CII was used to coat wells in 50 μl/well and the concentration of total immunoglobulin, IgG1 and IgG2 were determined by using #250, #182 and #183 detection reagents purchased from Pharmingen company.

In DBA/1 experimental mice, IgG1, an immuno protein which was derived from T-help 2 cell (Th2) and participated in the anti-inflammation, increased remarkably to above 2 times in the drug treated group compared with that in the control group. However, IgG2 presenting antigens in the direction Th1 [symbol] IgG2 for the inflammation induction and total immunoglobulin IgG maintained as it was. Namely, the treated group induced anti-inflammation specifically acting on Th2 [symbol] IgG2 among T cells (showed 115.5% increase of IgG1 concentration).

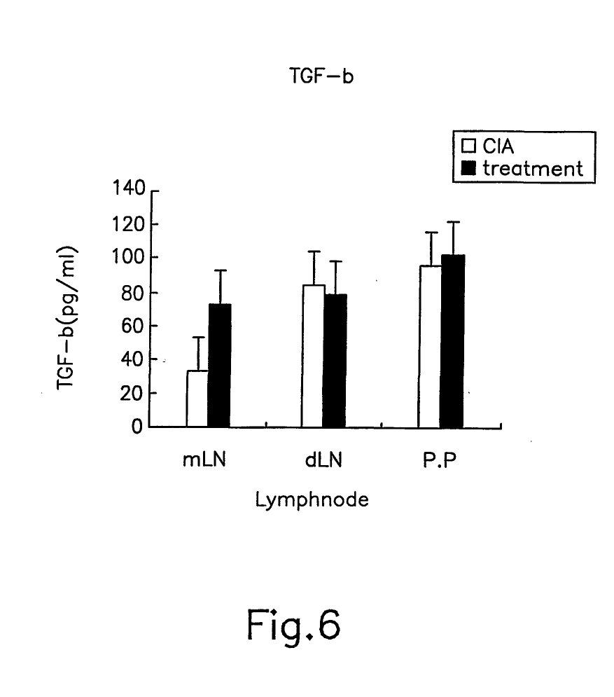

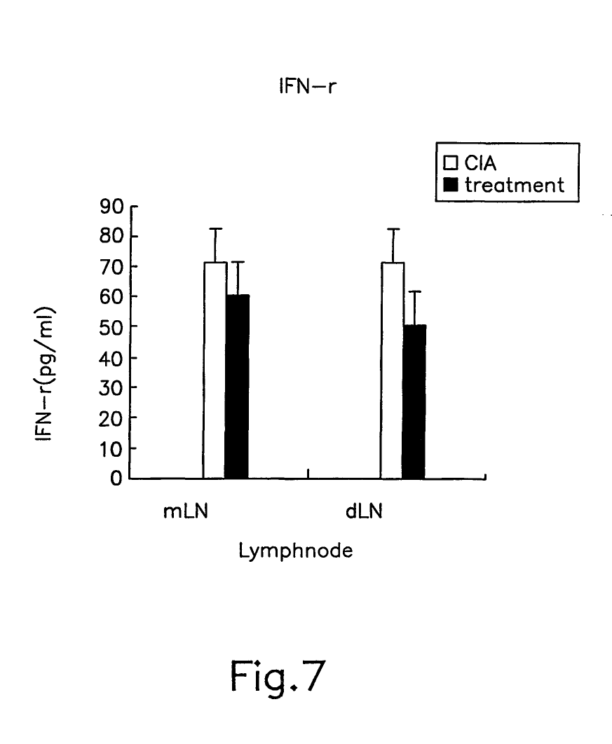

As described in FIG. 6 and FIG. 7, the intracellular concentration of TGF-β and INF-γ was determined in secondarily victimized mice cells which were ablated from various tissues by performing ELISA analysis. After 2 weeks of the booster, the TGF-β increase of the nodi lymphatici mesenterici in the tolerance group was identified to be more remarkable (approximate 2.5 fold) than that in the CIA group and the INF-γ value related with inflammation also reduced to 20˜40% in mLN and dLN of the drug treated group.

TGF-β existed within osteoblasts was accumulated inactively in bone matrix but activated by acids discharged from osteoclast during the bone absorption. Thus it can promote the proliferation of osteoblast, the collagen synthesis and the inhibition of osteoclast. In addition, TGF-β can be considered a monokine produced by macrophage but generally known to be produced in cells of bone or bone marrow and to activate epithelium and mesenchymal cells. Precisely, TGF-β showed anti-inflammation inhibiting MHC2 action (a kind of marker) of T cell.

Besides, the concentration of TGF-β, TNF-α and IL-4 were measured by performing RT-PCR method in 10 μg of mLN, dLN, P.P and spleen tissue cells and analyzed electrophoretically so that IOD values were obtained (unit amount: ng). Cellular RNA expressed was detected sensitively by using the RT-PCR method comprising the steps: separating specific RNA, synthesizing cDNA with the reverse transcription and amplifying with polymerase chain reaction and comparing the result with that in the control group and the detection curve for the analysis.

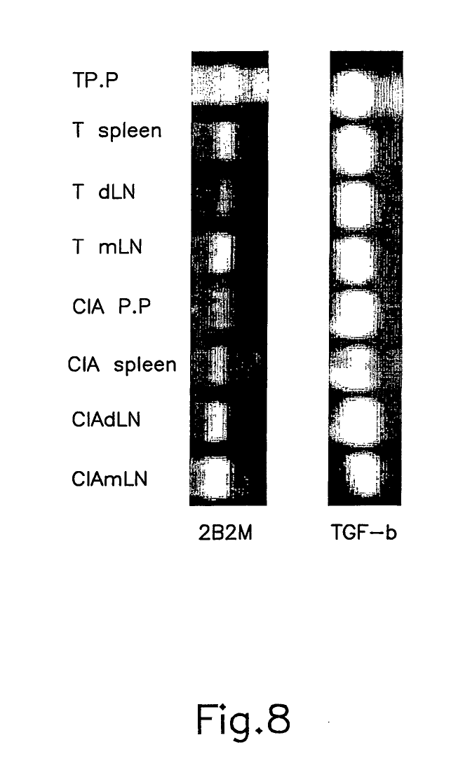

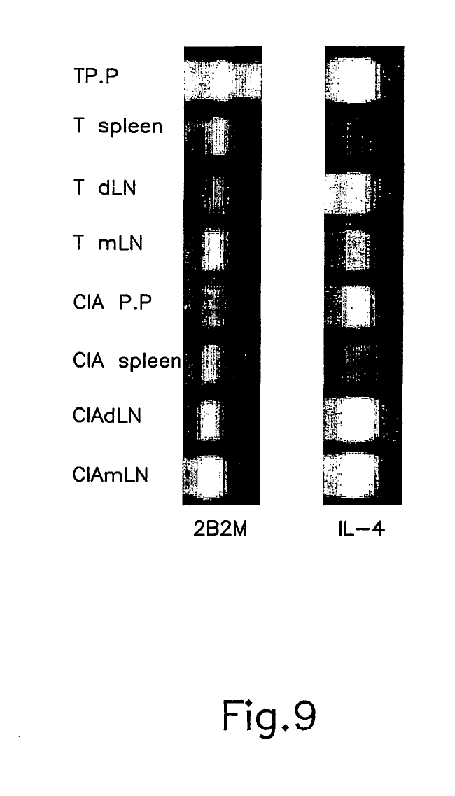

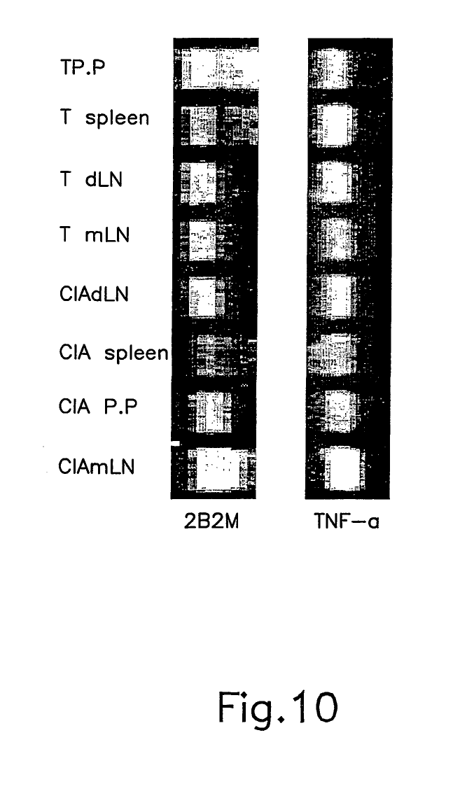

FIG. 8, FIG. 9 and FIG. 10 represents the intracellular TGF-β, IL-4 and TNF-α which were analyzed by performing PCR and electrophoresis. Besides, Table 7, Table 8 and Table 9 depicted IOD values measured by using the fluorescent detector at 450 nm.

TABLE 7
RT-PCR results of intracellular TGF-β in various tissues

| | mLN | | | dLN | | | Spleen | | | P.P | | |
|---|---|---|---|---|---|---|---|---|---|---|---|---|
| | β2M | Result | | β2M | Result | | β2M | result | | β2M | result | |
| CIA group | 20435 | 14852 | 0.73 | 14302 | 21081 | 1.47 | 6323 | 12230 | 1.93 | 8169 | 19263 | 2.36 |
| Drug | 7622.1 | 19310 | 2.53 | 4002.5 | 19668 | 4.91 | 5340.5 | 17939 | 2.36 | 3509.4 | 13957 | 3.98 |
| treated | | | | | | | | | | | | |
| group | | | | | | | | | | | | |

| | | | | |
|---|---|---|---|---|
| Difference | 247% increase | 234% increase | 74% increase | 69% increase |

TABLE 8
RT-PCR results of intracellular IL-4 in various tissues

| | mLN | | | dLN | | | Spleen | | | P.P | | |
|---|---|---|---|---|---|---|---|---|---|---|---|---|
| | β2M | Result | | β2M | Result | | β2M | result | | β2M | result | |
| CIA group | 20435 | 9653 | 0.47 | 14302 | 4161 | 0.29 | 6323 | 2364 | 0.37 | 8169 | 8697 | 1.06 |
| Drug | 7622.1 | 7404 | 0.97 | 4002.5 | 3469 | 0.87 | 5340.5 | 1237 | 0.23 | 3509.4 | 5447 | 1.55 |
| treated | | | | | | | | | | | | |
| group | | | | | | | | | | | | |

| | | | | |
|---|---|---|---|---|
| Difference | 106% Increase | 200% increase | 38% decrease | 46% increase |

TABLE 9
RT-PCR results of intracellular TNF-α in various tissues

| | mLN | | | dLN | | | Spleen | | | P.P | | |
|---|---|---|---|---|---|---|---|---|---|---|---|---|
| | β2M | Result | | β2M | Result | | β2M | result | | β2M | Result | |
| CIA group | 4953 | 15743 | 3.18 | 3537 | 9841.1 | 2.78 | 1737 | 10057 | 5.79 | 2496 | 7484.5 | 3.00 |
| Drug | 3379 | 6791.5 | 2.01 | 3329 | 8364.3 | 2.51 | 1908 | 10180 | 5.34 | 2680 | 5000.8 | 1.87 |
| treated | | | | | | | | | | | | |
| group | | | | | | | | | | | | |

| | | | | |
|---|---|---|---|---|
| Difference | 37% decrease | 10% decrease | 8% decrease | 38% decrease |

As described above, IOD value changes were calculated in the initial cDNA of β 2M and after the PCR amplification. The amount of TGF-β was detected to increase to 2.5 times in the booster site and in nodi lymphatici mesenterici, which supported the results of the above ELISA analysis and illustrated that TGF-β increased considerably in spleen and Peyer's patch and played a role to reduce inflammation and proliferate osteoclasts (See Table 7).

As a B cell (bone marrow cell) stimulating factor, IL-4 concentration also increased to 100˜200% in the booster site and in nodi Iymphatici mesenterici and showed that IL-4 can activate B cell vigorously and the concentration had not any difference in spleen, P.P and the like regardless of bone marrow cell (See Table 8).

Intracellular TNF-α was a tumor necrosis factor and related with the inflammation induction. As an experimental result, TNF-α concentration did not vary in spleen and drainic LN and reduced remarkably in mesenterium and P.P, which confirmed that TNF-α affected the transition and the differentiation of arthritis considerably. Thus the drug treated group had a very low ratio of arthritis induction unlike CIA induced group when boosted to measure the incidence (See FIG. 9).

### Experimental Example 4

In vitro Experiment of Mice Macrophges

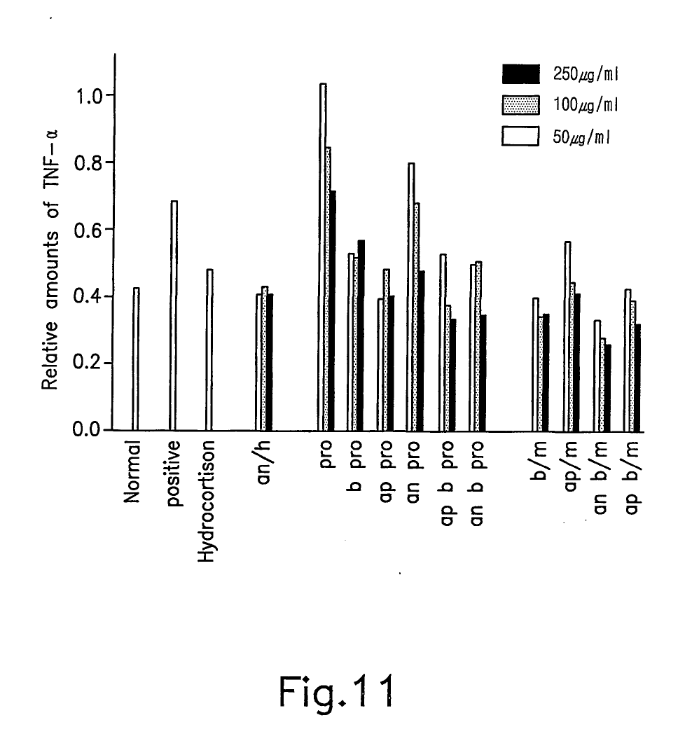

Among the herbal extract compositions prepared in Example 18, 11 samples and hydrocortisone as a comparative drug were examined to measure the inhibition effects of TNF-α derived from LPS stimulated mouse macrophage and collagenase. As a result, the ELISA experiment of TNF-α showed a statistically significant difference in the LPS group and the negative group and the fermented extract prepared during the fermentation procedure was depicted to have no effect. 2 components of this drug, Achyranthis roots (oosle) and Atractylodes japonica roots (changchul) were confirmed to have TNF-α inhibition effect which can be calculated to have remarkable inhibition effects when the weight deviations (Achyranthis roots 9.2 g [symbol] 23.1 g, Atractylodes japonica roots 12.2 g [symbol] 22.9 g) due to the fermented extract were substracted. This supported the results of the above Experimental Example 3 as described in FIG. 11.

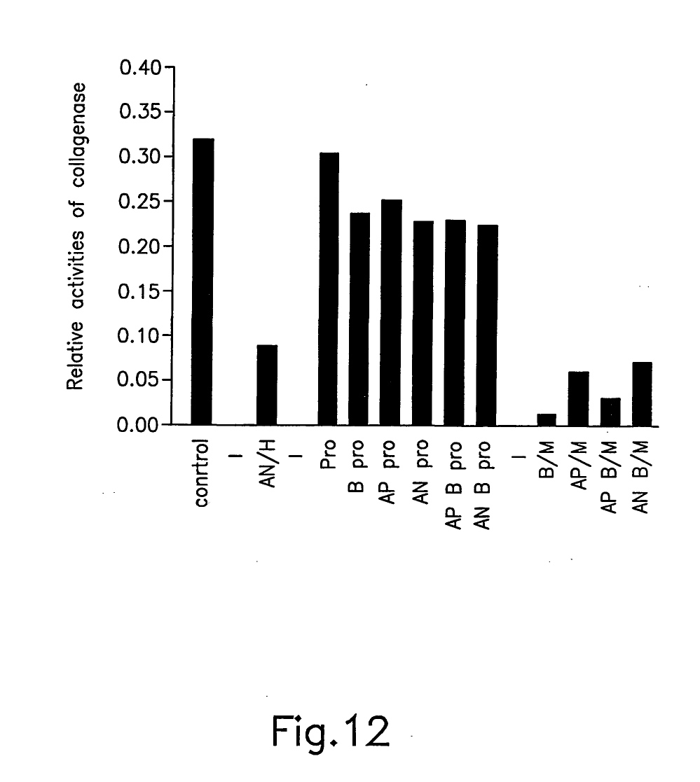

The collagenase inhibition experiment was performed by using extracts of Achyranthis roots and Atractylodes japonica roots, 10 samples of fermented extracts and the fermented solution itself and these results were measured for the comparison. Firstly, 0.5 ml solution containing 2% Azo dye impregnated collagen (Sigma company), CaCl₂ 1 nM, Tris-HCl 50 nM and collagenase type II (125 ng/0.5 ml) was reacted with each drug at 37° C. and for 15 hours enzymatically. Then at 540 nm, the amount of azo dye separated by the reaction was measured with UV/vis spectrophotometer and compared. Consequently, each herbal extract and these fermented products showed to have excellent effects for inhibiting collagenase activities as described in FIG. 11 and FIG. 12.

TABLE 10
In vitro experiments of collagenase inhibition.

| | Enzyme + drug | | activity in |
|---|---|---|---|
| name of drug | (average of 2 exp.) | drug | inhibitor |
| AN/B in MeOH | 0.329 | 0.256 | 0.073 |
| AP/MeOH | 0.32 | 0.258 | 0.062 |
| AP/B in MeOH | 0.2745 | 0.243 | 0.0315 |
| B MeOH | 0.2425 | 0.228 | 0.0145 |
| B pro in water | 0.516 | 0.278 | 0.238 |
| AP/B pro in water | 0.489 | 0.259 | 0.2 |
| AN pro in water | 0.515 | 0.285 | 0.23 |
| AN/B pro in water | 0.492 | 0.268 | 0.22 |
| AP pro in water | 0.5365 | 0.283 | 0.2535 |
| pro | 0.6075 | 0.304 | 0.3035 |
| AN in water | 0.3265 | 0.237 | 0.0895 |
| Negative control group | 0.319 | | |

Industrial Applicability

As described in the present invention, the herbal extract-based pharmaceutical compositions of Achyranthis roots (oosle), Atractylodes japonica roots (changchul) can alleviate the arthritis symptom partially, but their fermented products have more excellent effects for treating arthritis than those of non-fermented extracts in about 2 times.

In addition, the fermented herbal compositions of the present invention shows a very low ratio of arthritis transition during the drug administration for preventing and treating arthritis and are confirmed to prevent arthritis definitely. Besides, the arthritis induction also reduces to 67% in the efficacy. Consequently, the herbal extract compositions of the present invention are identified to be outstanding for treating arthritis and edema since they can regulate the concentrations of cytokines related with internal edema and arthritis definitely.

Those skilled in the art will appreciate that the conceptions and specific embodiments disclosed in the foregoing description may be readily utilized as a basis for modifying or designing other embodiments for carrying out the same purposes of the present invention. Those skilled in the art will also appreciate that such equivalent embodiments do not depart from the spirit and scope of the invention as set forth in the appended claims.

What is claimed is:

1. A process for preparing a herbal extract composition for preventing and treating arthritis, which comprises the steps of;
a) extracting Achyranthis roots and Atractylodes japonica roots; and
b) fermenting the said herbal extract.

2. The process for preparing the herbal extract composition according to claim 1, which comprises a further step; concentrating the extract after the fermentation step.

3. The process for preparing the herbal extract composition according to claim 1, in which the solvent of the said a) extraction step can be selected from the group consisting of distilled water, ethanol, methanol, propanol and butanol.

4. The process for preparing the herbal extract composition according to claim 1, in which the ratio of Achyranthis roots and Atractylodes japonica roots is 0:5˜5:0.

5. The process for preparing the herbal extract composition according to claim 1, in which the Achyranthis roots and the Atractylodes japonica roots are utilized in dried state or in raw state.

6. The process for preparing the herbal extract composition according to claim 1, in which the Achyranthis root is Achyranthis japonica (MIQ) NAKAI or Achyranthis bidentata BLUME and the Atractylodes japonica is Atractylodes japonica KOIDZ or Atractylodes Koreana Kitam.

7. The process for preparing the herbal extract composition according to claim 1, wherein substrate adopted for the fermentation step can be one or more two selected from the group consisting of malt, malted wheat, grape and wine fermentation enzyme source and yeast; and as a by-substrate for the fermentation, cooked rice or rice, corn, starch, wheat, papain, kiwi or pear can be utilized directly or in refined forms excluding starch, or one or more than two selected among their glucose syrups can be contained with 0˜50%.

8. The process for preparing the herbal extract composition according to claim 1, in which for the fermentation step, substrate, by-substrate and co-enzyme agent are added into the herbal extracts directly or they are mixed after each of the components is prepared separately.

9. The process for preparing the herbal extract composition according to claim 1, in which for the b) step, the fermentation condition is 2˜36 hours at 20˜75° C.

10. A herbal extract composition, which is prepared by using the process of any one selected from claims 1˜9.

11. The herbal extract composition, which is prepared by using a) step of claim 1 and contains Achyranthis roots extract, Atractylodes japonica roots extract or their mixture.

12. The herbal extract composition according to claim 10 or claim 11, which takes an oral administration form selected from the group consisting of tablets, capsules, caplets, syrup, pills, liquid solutions, suspensions, powders, lozenges, micronized particles and osmotic delivery systems.

13. The herbal extract composition according to claim 12, which comprises the fermented extract as a major pharmaceutical component and pharmaceutically effective and acceptable carrier by using a proper vehicle.

14. The herbal extract composition according to claim 10, which enhances the production of transforming growth factor β (TGF-β) in the body.

15. The herbal extract composition according to claim 10, which inhibits the production of interferon γ in the body.

16. The herbal extract composition according to claim 10, which inhibits the production of tumor necrosis factor α (TNF-α) in the body.

17. The herbal extract composition according to claim 10, which enhances the production of interleukin-4 (IL-4) in the body.

18. The herbal extract composition according to claim 10, which enhances the production of mouse immunoglobulin G1.
